(19) United States
(12) Patent Application Publication
(10) Pub. No.: US 2023/0284974 A1
(43) Pub. Date: Sep. 14, 2023

(54) SYSTEMS AND METHODS FOR DIAGNOSING, ASSESSING, AND QUANTIFYING SEDATIVE EFFECTS

(71) Applicant: Weneuro Inc., Tel Aviv (IL)

(72) Inventors: Wayne Dam, Port Coquitlam (CA); Zeev Roich, Tel Aviv (IL)

(21) Appl. No.: 18/119,159

(22) Filed: Mar. 8, 2023

Related U.S. Application Data
(60) Provisional application No. 63/318,267, filed on Mar. 9, 2022.

(51) Int. Cl. A61B 5/00; A61B 5/11

(52) U.S. Cl. CPC A61B 5/4848; A61B 5/1114; A61B 5/74; A61B 5/4064; A61B 5/0077; A61B 5/4806; A61B 5/4845

(57) ABSTRACT

Systems and methods are described for diagnosing, assessing, and quantifying sedative effects in a subject. The systems and methods described herein are non-invasive and based on the detection, measurement, and analysis of involuntary micromotions and/or fixational eye movements with respect to a subject's eyes, pupils, and head. Determinations as to sedation are based, in part, on divergences between measurements associated with the subject and predefined ranges, threshold values, and/or measurements contained in one or more individualized or population datasets. The described systems and methods are not reliant on the cooperation of the subject and, as a result, do not interfere with the subject's ongoing activities. The systems and methods can also be continuously deployed over any period of interest, and the results can be predictive in nature as opposed to reactionary.

This non-provisional application claims benefit of priority to U.S. Provisional Patent Application No. 63/318,267, titled “SYSTEMS AND METHODS FOR DIAGNOSING, ASSESSING, AND QUANTIFYING SEDATIVE EFFECTS” and filed Mar. 9, 2022, which is incorporated herein in its entirety for all purposes.

### BACKGROUND

The present disclosure relates generally to the field of diagnosing, and assessing the extent of, sedative effects (e.g., intoxication) and/or drowsiness in a subject. In many cases, it is important to monitor sedative effects in an individual in a non-invasive manner and promptly alert the subject and/or other individuals as soon as possible once sedative effects are observed. For example, in the course of their jobs, professional truck drivers or airline pilots spend a considerable amount of time and many consecutive hours in a seated position. However, it is critically important that these drivers and pilots remain alert at all times as even a short lapse in alertness, responsiveness, or consciousness could lead to disaster and place lives at risk.

Today, clinical diagnosis and assessment of sedative effects such as those brought on by alcohol intoxication require the use of tools such as a breathalyzer that assesses the concentration of alcohol in a subject's breath. Such tools suffer from many drawbacks, including the fact that the tools only detect intoxication (rather than a broader range of sedative effects such as sleepiness, for example) and require the subject to perform actions that may pause or otherwise interfere with the subject's ongoing activity.

Other tools for maintaining the alertness of a subject (for example, a driver of a vehicle) rely on providing stimulus to the subject and, in some cases, require responses from the subject. For example, systems exist that may periodically prompt a subject to speak a word or touch an input button within a specified amount of time. Such systems may conclude that if the subject's response is received outside a predetermined amount of time, the delay can be attributed to drowsiness or other sedative effects in the subject.

Such systems are highly subjective in nature. For example, depending upon the nature of the subject's ongoing activity, an immediate response to the provided stimuli may not have been possible or safe. For instance, in the case of a driver or pilot, the individual may be performing a maneuver or task at the time of the system's prompt that prevents the individual from providing the requested response within the predetermined amount of time. In such cases, the system may report that the subject is suffering from sedative effects when no such effects are present. Alternatively, a subject may be suffering from sedative effects, such as on the verge of falling asleep, and still provide the requested feedback (e.g., voice or physical input) within the predetermined amount of time. In such cases, the system will incorrectly conclude that the subject is alert.

As a result, a need exists for new and improved tools for diagnosing and assessing sedative effects in a subject. In particular, a need exists for new and improved systems and methods that test for a broad spectrum of sedative effects (e.g., drowsiness and intoxication), do not interfere with a subject's ongoing activities, and do not require voluntary responses from the subject. A need further exists for testing equipment and diagnostic tools that provide more objective outputs than existing tools, cannot be circumvented by the user/subject, and are predictive in nature as opposed to reactionary (i.e., only alerting the subject or other individuals after a subject has fallen asleep or otherwise become unconscious). A need also exists for testing equipment and diagnostic tools that can provide continuous monitoring of a subject rather than one-time, sporadic, or periodic monitoring.

### SUMMARY

Examples described herein include systems and methods for diagnosing, assessing, and quantifying sedative effects in an individual including, for example, the evaluation of drowsiness and/or intoxication. In particular, the systems and methods described herein are non-invasive and based on the detection, measurement, and analysis of involuntary micromotions and/or fixational eye movements with respect to an individual's eyes, pupils, and head. These systems and methods are aimed at solving the problems and drawbacks associated with existing diagnostic processes and equipment (e.g., breathalyzers and prompting systems) and mitigating the risk of delays between the onset of sedative effects and the issuance of an alert or notification to the subject or other individuals. These systems and methods are further aimed at improving upon diagnostic techniques that measure voluntary movements or feedback of subjects, including but not limited to spoken responses and voluntary movements of the subjects' eyes, head, or limbs.

In examples, systems and methods described here include recording, by a camera, one or more videos of at least a portion of a subject's face. Within the one or more videos, one or more biomarkers associated with the subject can be identified. In further examples, once identified, involuntary micromotions or fixational eye movements associated with the biomarkers are measured over the duration of a testing period (e.g. between 20 msec and 7000 msec). In some examples, these measurements are compared to measurements from a dataset in order to determine whether any divergences exist between the biomarker measurements and the dataset. Divergences, whether between a subject's current and past measurements or between a subject's current measurements and those of a population group, can indicate sedative effects. Conversely, the lack of a divergence between the same data can indicate the absence of sedative effects. In a further aspect, the lack of a divergence between the subject's measurements and those of a population group having confirmed sedative effects (e.g., intoxicated or sleep deprived) can be indicative of sedative effects in the subject.

The examples summarized above can each be incorporated into a non-transitory, computer-readable medium having instructions that, when executed by a processor associated with a computing device, cause the processor to perform the stages described. Additionally, the example methods summarized above can each be implemented in a system including, for example, a memory storage and a computing device having a processor that executes instructions to carry out the stages described. As used herein, the terms “computing device,” “user device,” and “mobile device” are interchangeable and can encompass any type of computing device, such as laptops, tablets, smart phones, and personal computers.

Both the foregoing general description and the following detailed description are exemplary and explanatory only and are not restrictive of the examples, as claimed.

### BRIEF DESCRIPTION OF THE DRAWINGS

FIG. 1 is a flowchart of an example method for diagnosing and assessing sedative effects in a subject.

FIG. 2 is an illustration of an example system for diagnosing and assessing sedative effects in a subject.

FIG. 3 depicts an example graphical user interface for diagnosing and assessing sedative effects in a subject.

FIG. 4 depicts an example graphical user interface for diagnosing and assessing sedative effects in a subject.

### DESCRIPTION OF THE EXAMPLES

Reference will now be made in detail to the present examples, including examples illustrated in the accompanying drawings. Wherever possible, the same reference numbers will be used throughout the drawings to refer to the same or like parts.

Applicant's systems and methods are aimed at solving the problems associated with detecting sedative effects in an individual, such as intoxication and sleep deprivation or drowsiness. The systems and methods can be used both for initial diagnosis and/or for continuous monitoring of an individual over a period of time or the duration of a task (e.g, driving a truck or flying a plane from one location to another).

Using the disclosed systems and methods, the user can self-assess the presence and/or degree of the effect of drowsiness or substances on the central nervous system using the systems and methods. The disclosed systems and methods can also be used by another individual to assess the presence of sedative effects on the central nervous system in another person, regardless of whether the individual performing the monitoring is present with the subject or remotely located. In one aspect, no special medical qualifications are required to use the systems and methods. In another aspect, the systems and methods can also provide objective and personalized results without requiring or assessing the voluntary movements of a subject during testing. In a further aspect, the systems and methods can perform an identification of the user based on their biometric data simultaneously with the assessment of sedative effects.

Sedative substances interact with brain activity causing it to slow down. Sleep deprivation or drowsiness can have a similar effect. The systems and methods described here can collect and/or measure motion associated with a number of biomarkers of a subject and detect divergences in biomarker motion between the subject's current state and a dataset that, for example, contains historical biomarker motion for the subject at a time when sedative effects were not present. In another example, the dataset can contain historical biomarker motion indicating a lack of sedative effects in a population group.

As used herein, “biomarkers” can be involuntary micromovements of an individual's eyes or head, including but not limited to ocular drift and tremor (e.g., micro-tremors of the pupil), oscillation and/or change in pupil or iris size, microsaccades (as opposed to voluntary saccades), oscillation of regulation of blood vessels (e.g., facial blood vessels), and oscillation of muscle tone. In some embodiments, the measurements are directed to fast processes and short oscillations such that the measurement process can be completed in under 10 seconds. In further embodiments, the measurements process can be completed in approximately 7 seconds or less. In still further embodiments, the measurements process can be completed between 20 msec and 7000 msec. Detected movements, in such embodiments, do not rely on saccades or smooth pursuit with respect to eye movements, both of which result from voluntary movement of the subject and require a testing sequence that requires more time to measure.

In one aspect, decreased activity of the central nervous system associated with sedative effects can correlate with changes in the properties of an individual's involuntary micromotions and/or fixational eye movements. For example, fixational eye movements can be observed in a range above approximately 4 Hz with angular amplitude up to approximately +\−0.3 degrees. Suppression of central nervous system activity is accompanied, generally speaking, by a shift of oscillation frequency to a lower range and\or reduction of oscillation power. In some embodiments, any suitable system or method of analysis of the oscillations can be used.

The transition between alertness and sleepiness or intoxication can correlate with a change in activity of the central nervous system. Thus, Applicant's systems and methods can be used to detect and assess a user's sleepiness or intoxication.

In another aspect, the measurements made by the systems and methods can be performed either in a passive form (e.g. video recording of a face) or in an active form (e.g., “live” video imaging). In the latter case, in some examples, the system can prompt the user to perform an action. In some examples, the system can prompt the user or affected individual to look at their hand in front of them (or another object in close proximity) or a distant object such as a building across the street. While some of these prompts may result in voluntary movements of the subject, the biomarkers of the subject that are measured during the test may still be involuntary micromotions and/or fixational eye movements. Any significant divergence between the present properties of the individual's micromotions or fixational eye movements and a dataset associated with an alert individual (whether the subject or a population group) can be a sign of sedation. The degree of divergence (or lack thereof) can be assessed either by comparison with, for example: (1) a previous measurement for the subject taken at a time when the subject is confirmed to be alert and unimpaired; (2) by comparison of the subject's measurements with population data from, for example, a database comprising data associated with alert and/or unimpaired individuals; or (3) by comparison of the subject's measurements with population data from, for example, a database comprising data associated with sedated, intoxicated, or otherwise drowsy individuals. In another aspect, the population data can be derived from a population segment of similar age, gender, prior health conditions, environment, etc. as the subject.

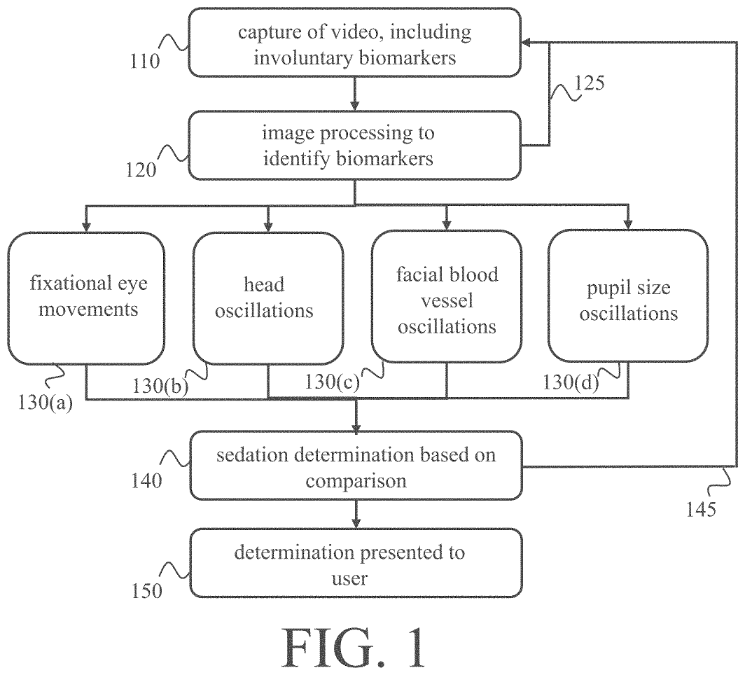

FIG. 1 depicts a process for diagnosing, assessing, and quantifying the presence of sedative effects based on a subject's involuntary micromotions and/or fixational eye movements with respect to an individual's eyes and/or head. In one aspect, at step 110, a computing device (described in more detail below) is used to record a video of an affected individual's face. The recording can be performed by a computing device (e.g., smartphone or laptop) with a suitable camera, processor, and memory. In some embodiments, the video includes the individual's head and face. In other embodiments, the video includes the individual's eyes and the skin or tissue around the eyes. In another aspect, a “live” image of the camera's view is presented to the user at a display of the computing device to help the user determine where and how to position the camera to adequately capture the user's eyes or head.

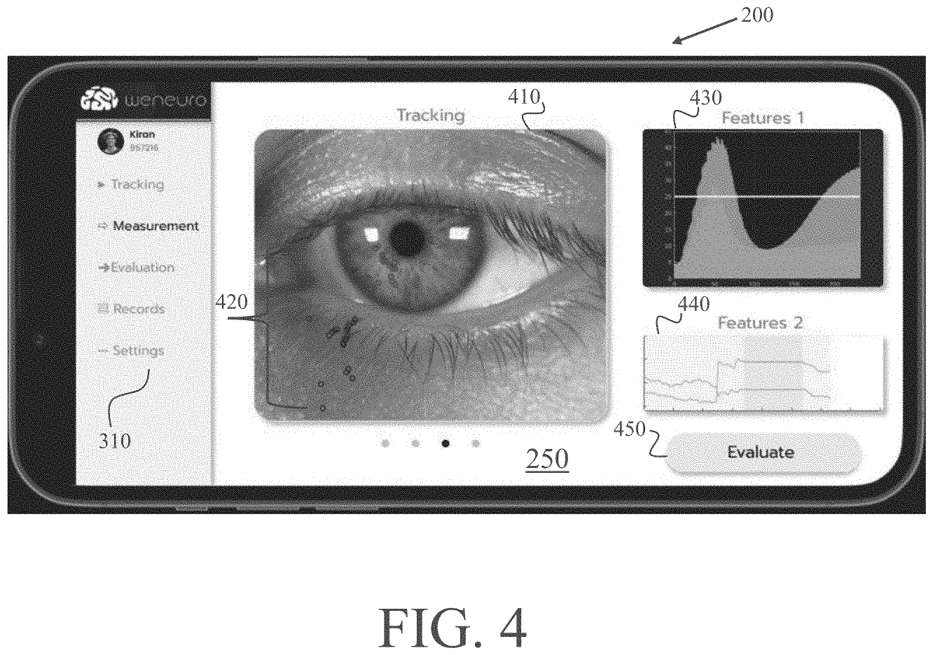

At step 120, the recorded image can be processed by the computing device. In one aspect, in order to minimize variables introduced by a subject's participation in the diagnosis, a set of involuntary biomarkers or image features are selected for measurement. The biomarkers contained in the recording can then be identified within the video (e.g., the biomarkers 420 identified in image 410 of FIG. 4) and tracked as time elapses (e.g., between 20 msec and 7000 msec). In some embodiments, the involuntary biomarkers can include, but are not limited to, fixational eye movements (e.g., microsaccades, drift, or tremor) and/or oscillations of the subject's head, facial blood vessels, facial muscle tone, or pupil size with respect to isolated locations identified in the subject's eye(s) (depicted, in one example, in FIG. 4, items 420).

At step 130, in some examples, the Kanade-Lucas-Tomasi (“KLT”) feature tracker can be used to track image features (e.g., biomarkers) within a recorded video. For motion magnification, in some embodiments, a two-dimensional dual-tree complex wavelet transform can be used. The data collected with respect to the selected biomarkers over the duration of a test period (e.g., between 20 msec and 7000 msec) can then be analyzed. In further examples, the collected data can include involuntary movements such as XY fixational eye movement, changes in pupil size, and oscillations associated with the subject's head, facial blood vessels, or facial muscle tone.

In still further examples, at step 130(a), fixational eye movements (e.g., microsaccades, drift, and/or tremor) associated with the subject are measured and, based on the measurements, divergences from a dataset can be assessed. In these examples, “divergence” is used to describe divergences: (1) between a subject's present biomarkers and the subject's biomarkers recorded in a previous test; or (2) between a subject's biomarkers and the biomarkers of a population group. In any case, divergences in the measured velocity and amplitude of fixational eye movements can be indicative of sedative effects, including but not limited to intoxication, sleep deprivation, and/or drowsiness. For example, a significant divergence (e.g., p<0.001) of microsaccades velocity and amplitude can be indicative of sedation. In other embodiments, a divergence in eye drift and tremor can be indicative of sedation. For example, comparing drift in a subject's right eye before and after alcohol intoxication or the onset of some other sedative effect can result in a decrease of mean displacement in 5-10 Hz from approximately 1.92±0.06 deg to approximately 1.71±0.05 (p<0.01) and in 10-20 Hz from approximately 1.43±0.05 deg to approximately 1.27±0.04 (p<0.01). In another example, comparing tremor in a subject's right eye before and after alcohol intoxication or the onset of some other sedative effect can result in a mean ratio of displacement in 40-50 Hz/70-80 Hz decreased from approximately 1.88±0.03 to approximately 1.76±0.02 (p<0.0001). These examples are only illustrative and similar results can be found in a subject's left eye, for example.

In another aspect, at step 130(b), head oscillation in the subject is also (or alternatively) recorded and tracked. In one aspect, accounting for head movement allows for the isolation of eye movement(s) because movement of the head can be accounted for and removed. In another aspect, at step 130(c), oscillations in the subject's facial blood vessels can be recorded and tracked. In one aspect, small changes in skin color which can be detected by the camera are sufficient to detect flowing blood in facial blood vessels. Using video plethysmography, for example, involuntary micromotions associated with blood volume pulsations in facial tissue can be detected and measured over the testing time period. Changes or divergences in such micromotions can be indicative of sedation.

In another example, at step 130(d), oscillations in pupil size of the affected individual's right and/or left eye over the course of the testing period can be analyzed. The size of an individual's pupils can change continuously, for example, in response to variations in ambient light levels. This process is modulated by cognitive brain function and the onset of sedation can cause a change in how an individual's pupils respond to changes in ambient light. In some examples, significant divergences between a subject's maximum pupil diameter, papillary recovery time, constriction velocity, and latency of the pupillary light response can be detected and measured. Divergences exceeding a determined threshold can be indicative of sedation. Ambient light and any changes thereto over the course of a test can also be measured by, for example, data received from a light source (e.g., a flash or flashlight of a smartphone) or data derived from the recording of the individual's face (e.g., brightness and/or changes to skin tone of the recorded individual). Any such changes in ambient light can then be accounted for when assessing oscillations in pupil size or other fixational eye movements.

In some embodiments, the unit of measurement for the movement of tracked image features (e.g., biomarkers 420 in FIG. 4) within a recorded video is degrees per time period (e.g., 5° in 2 seconds or, in another example, 3°/second). Eye movement can be measured as a rotation of the eyeball within the subject's head so rotation of the eyeball can be measured in degrees. In further embodiments, tracked image features (e.g., biomarkers 420 in FIG. 4) can be tracked as pixels identified and followed in an image/recording. In one example, if the distance between the camera and the eye is known, a pixel in the image/recording can be converted to, for example, millimeters (if XY movement over time is the unit of measurement) or degrees (if rotational movement over time is the unit of measurement).

As described here, any of steps 130(a)-(d) can be executed to the exclusion of any of the other steps. Similarly, any of steps 130(a)-(d) can be executed together, simultaneously, or in addition to any of the other steps.

At step 140, regardless of which biomarkers are identified, measured, and analyzed, the computing device can analyze the measured biomarker movements based on frequencies and power of oscillations (e.g., iris oscillations). In some examples, a shift of oscillation frequency to a lower range and\or a reduction of oscillation power can indicate an increase or higher likelihood of a sedative effect. Similarly, a shift of oscillation frequency to a higher range and\or an increase of oscillation power can indicate a decrease or lower likelihood of a sedative effect.

In some embodiments, the measured results associated with the subject's biomarker movements can be compared to predefined ranges or stored values to assess the presence of a sedative effect. In other embodiments, the measured results can be compared to a dataset to determine the presence or absence of sedation. For example, the subject's biomarker data can be compared to a database comprising historical data associated with the subject. In one example, the collected biomarker data can be compared to historical data associated with the subject at a time when it was confirmed the subject was not sedated, drowsy, or otherwise impaired. For instance, in a case where the subject is engaged in an hours-long drive, the subject's biomarker data from a present test can be compared to the subject's biomarker data collected before the drive or at a time earlier in the drive.

In other examples, the subject's biomarker data can be compared to a database comprising historical data associated with a population group. In some instances, the historical data can be associated with a population group that is free of any sedative effects, a population group suffering from sedative effects, or some a combination of the two. A divergence between the measured biomarkers of the subject and those associated with individuals free of sedative effects can be indicative of sedation. For example, a divergence in the subject's biomarker(s) outside a predetermined value such as one or two standard deviations from such a population group can be indicative of sedation. Conversely, a convergence (or lack of divergence) between the measured biomarkers of the subject and those associated with individuals suffering sedative effects can also be indicative of sedation in the subject.

At step 150, the results of the analysis can be presented to the user via a user interface (“UI”) located at the computing device. In some examples, the presentation can include an indication of whether sedative effects are present and, in appropriate circumstances, remedial or corrective action can be taken. For example, where it is detected that the subject is drowsy or the subject's measurements are consistent with impending sleep or loss of consciousness, an alert can issue to the subject (e.g., siren and/or flashing lights) or a third party. In other examples, where intoxication is indicated, an alert can issue to the subject and/or a third party and the systems and methods described here can automatically intervene in the activity by, for example, cutting power to a vehicle in operation by the subject after a predetermined period of time (assuming such action can be taken safely). In further examples, the UI can include an indication of the severity of the sedation and suggest remedial actions (e.g., stretching by the subject or engaging the subject in a word game that requires the subject's interaction).

In further examples, the testing may not be able to proceed or the analysis may be inconclusive based, at least in part, on conditions present during the testing period (e.g., between 20 msec and 7000 msec). For example, ambient lighting during the testing period may have been inadequate to accurately detect, measure, or analyze one or more biomarkers. Alternatively, or additionally, the computing device may have detected physical movement of the camera (e.g., using an accelerometer present in the computing device) during the testing period that exceeds an acceptable threshold. In such cases where it is determined at step 120 that the testing conditions are inadequate, at step 125, instructions can be presented to the user via the UI to repeat the test under more favorable conditions. For example, the UI may instruct the user to move to a location with more ambient light or to perform the test again without moving the computing device during the test. In other examples, at step 125, the UI may instruct the user to repeat the test and prior to doing so, the computing device can automatically take remedial action(s) to resolve any problem(s) associated with the first test. For instance, in a situation where detected ambient light during the initial test is insufficient to ensure accurate test results, a flashlight or flash of the computing device may be instructed to activate and/or increase its intensity or brightness during a subsequent test.

In another aspect, if or when testing results at step 140 are inconclusive or additional imaging or data is needed, at step 145, instructions can be presented to the user via the UI to repeat all or a portion of a test and/or initiate a different testing protocol.

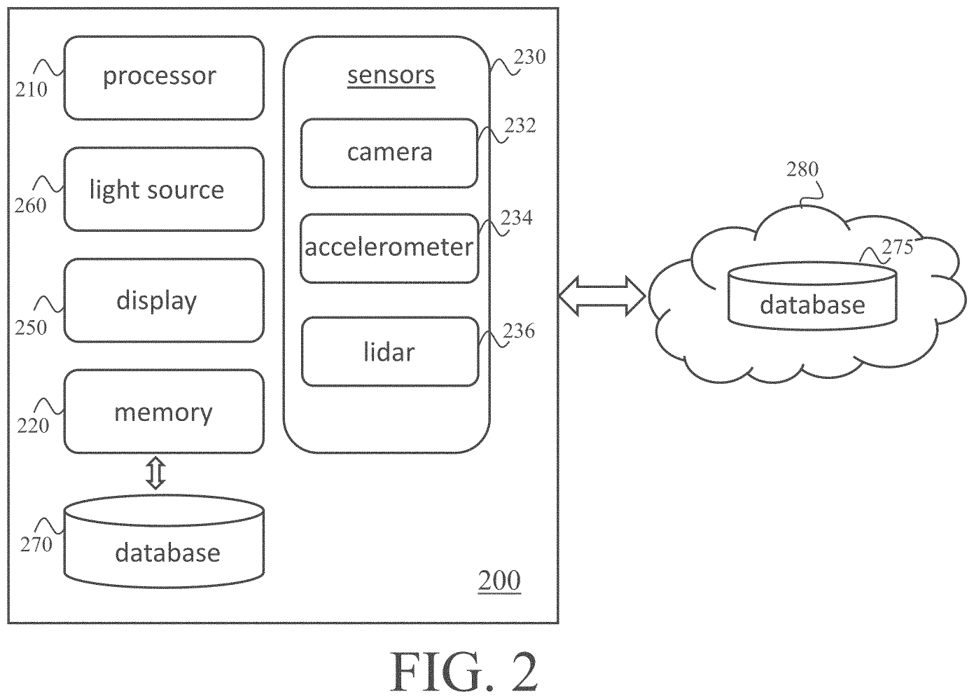

FIG. 2 depicts an example user device 200 for performing the processes described here. In some embodiments, the user device can comprise one or more of a processor 210, memory 220, sensors 230, and a display 250. The user device can be configured to measure the properties of a subject's right and left side (e.g., right and left eye), together and/or separately.

In one aspect, computing device 200 can contain one or more main sensors designed to detect a subject's fixational eye movements and one or more auxiliary sensors designed to make adjustments to the main sensors. For example, in some embodiments, computing device 200 can include a main sensor including a camera 232 for recording video of a subject (e.g., a video of the subject's iris) and an auxiliary sensor including a lidar 236 to measure the distance from the main sensor or camera to the user's eye. In such examples, the distance to the eye can be used to analyze the iris movements with higher accuracy.

In another aspect, computing device 200 can utilize one or more sensors to perform non-eye movement filtering (“NEMF”) and feature computation. In some embodiments, NEMF can be performed using one or more facial features to recognize a subject's head and/or hand movements during the image recording (e.g., the subject's eyelashes can be used to recognize the eye movements against the head movements). In such embodiments, NEMF can be used to filter out the movements of the image which do not relate to the subject's eye(s). For example, NEMF can filter out movements of the user's head or movements of the user's hand holding computing device 200.

In further embodiments, the NEMF can use any suitable objects or traits of the subject's face that maintain a known relationship with respect to the subject's eye(s). For example, NEMF can identify objects or traits of the subject's eyelashes or the nevus on skin next to the eye, etc. In still further embodiments, NEMF can process the movements of the object or trait to “subtract” them from calculations, i.e., help isolate movements attributable to the subject's eye(s). In other examples, the auxiliary sensors of computing device 200 can include an accelerometer 234. Accelerometer 234 can detect a user's hand movements during image recording (in instances where the user is holding computing device 200) and, as part of the NEMF described above, take such movements into account during subsequent analysis and to improve accuracy.

In another example, computing device 200 can include an alternative (or additional) accelerometer 234 placed proximate or close to the subject's eye, on the subject's eyelid, or directly on the subject's cornea for detecting tremors related to eye movements. In another example, computing device 200 can include a lidar 236 for measuring small movements of the user's cornea. In addition to sensors 230, in some embodiments, computing device 200 can further comprise a light source 260 such as a flash or flashlight.

In use, a user can hold computing device 200 in a way that the subject's face is visible to camera 232. In some embodiments, display 250 can depict instructions for the user that guide the user in positioning computing device for a test. For example, display 250 can instruct a user to move computing device 200 toward or away from the subject in order to adequately capture the appropriate biomarkers. In other examples, display 250 can instruct the user to move to an area with more or less ambient light. In still further examples, computing device 200 can instruct the user to steady the computing device in response to output from accelerometer 234 indicating the computing device is moving. Alternatively, in other embodiments, computing device can be mounted somewhere (e.g., the dashboard of a vehicle) and positioned so as to adequately capture the subject's face and/or eyes.

In another aspect, computing device 200 may automatically adjust in response to feedback from one or more sensors. For example, where computing device 200 detects too little ambient light in a recorded image from the camera, computing device 200 may instruct light source 260 to activate or increase in brightness. In another example, where accelerometer 234 indicates movement of computing device 200, the computing device's movements can be accounted for, and differentiated from, the subject's head and/or eye movements such that the subject's head and/or eye movements can be isolated. In this way, it is possible for head or eye movement to be recognized and measured even where computing device 200 or the subject's head is also moving.

Regardless of which biomarkers are identified, measured, and analyzed, computing device 200 may compare the measured biomarkers against historical data from a database 270. In some examples, the historical data corresponds with a population group comprising biomarkers for alert/non-sedated individuals, sedated individuals, or a combination of the two. In other examples, additionally or alternatively, database 270 can include historical data associated with the subject. For instance, the database can include historical data associated with the subject at a time when it was confirmed the subject was not sedated, intoxicated, sleep deprived, or otherwise drowsy.

In another aspect, to improve the quality of assessments, computing device 200 can remove or exclude significant eye movements and/or saccades exceeding some threshold, for example, movements which are greater+−0.1 degree. Alternatively or additionally, computing device 200 can remove or exclude trends and/or outliers in iris movements, for example, related to eye drift.

In another aspect, computing device 200 may be in communication with a network 280 such as the Internet. In such examples, some or all of the processing of biomarker information, including any comparisons between measured biomarkers and historical individual and/or population data, can be performed remotely (e.g., in the cloud). In other examples, some or all of the data within database 270 may be stored in remote database 275 in order to conserve memory space at computing device 200.

Whether some or all data is stored locally or in the cloud, and whether some or all of the processing of biomarker information occurs locally or in the cloud, in further examples, computing device 200 can further be configured to automatically alert a third party, such as emergency personnel or an employer, when test results indicate sedation or intoxication. Such an alert can include an indication of the severity of the subject's sedation, the user's location, and/or recommended remedial actions for the subject, for example.

In some embodiments, computing device 200 can receive input from sensors 230, including camera 232, evaluate measurements associated with one or more biomarkers in real-time, and provide real-time recommendations to the user via display 250 regarding the test results and/or how to perform or improve a subsequent measurement. For example, computing device can test left and right parts of the body independently (e.g., firstly, the right eye, secondly, the left eye). Computing device 200 can use the features of the images to adjust and/or compare two or more independent images (e.g., computing device 200 can use the sizes of the pupils or irises recorded for the left and right eyes to adjust the images to each other considering their sizes must be equal). In some embodiments, this can help to assess the distance between the camera and the user's face if auxiliary sensors such as lidar are not available or do not provide sufficient accuracy under the circumstances.

In some embodiments, computing device 200 can use any facial features to recognize head and/or hand movements during the image recording. For example, eyelashes can be used to recognize the pupil or iris movements against the head movements. The features can be the properties of the images in temporal and special domains. For example, color changes of the user's face within a period of time. In further or alternative embodiments, computing device 200 can also identify inconclusive tests, then computing device 200 can inform the user via display 150 and make suggestions to the user to perform additional measurements or tests.

In another aspect, display 250 can depict a UI used to guide a user through a test. In further examples, the UI can display step-by-step instructions for the user to carry out the test and, upon completion, display test results and/or remedial actions to the user. In the event of an inconclusive test or unfavorable testing conditions, the UI can also provide the user feedback for improving the test conditions and repeating the test.

The following examples describe tests that can be performed using computing device 200:

### Example 1

In some embodiments, the user may desire to detect whether they or another individual is under the influence of a substance or becoming drowsy. Using the UI of computing device 200, the user can open an application configured to assess sedative effects in a subject. The UI can suggest or prompt the user to aim camera 232 of computing device 200 at the subject's face or eye(s).

In such examples, a processing unit (“PU”) of computing device 200 can recognize in real-time the image of the subject's eye and/or face. The PU, together with camera 232, can record the image and perform preprocessing steps, including assessing the image quality and/or feasibility of biomarker extraction related to the left and/or right sides of the subject's face and whether involuntary micromotions and/or fixational eye movements associated with the biomarkers can be tracked and measured.

In some examples, if the PU determines that biomarkers cannot be extracted from the current image with a required or threshold level of accuracy (e.g., the PU cannot detect an eye on the image), in some embodiments, the PU can convey instructions, via the UI, to the user to bring computing device 200 closer to the subject's face or eye(s), or place computing device 200 on a steady surface (e.g., a table or tripod) to reduce interference due to possible movement (e.g., a tremor of the user's hands). The instructions can be visual (e.g., icons, text, lights), audio (e.g., voice, beeps, or sounds), and/or tactile (e.g., vibrations). Additionally or alternatively, the PU can provide instructions to other components of computing device 200 to correct image quality issues. For example, the PU can activate light source 260 and/or increase its brightness to more adequately illuminate the subject's face or eye(s).

In some embodiments, as part of an iterative process, the PU can record the image and again assesses its quality. If the quality is still insufficient, the PU can provide additional instructions to the user via the UI regarding how to properly take a measurement and/or take automatic steps to enhance the image quality.

Once the image quality is determined to be of sufficient quality, the PU can then process the image to isolate involuntary micromotions, oscillations, and/or fixational eye movements associated with the subject's biomarkers and compensate for any movements of the user's hands or the subject's head.

Regardless of the specific biomarker(s) measured, the PU can compare the measurements to predetermined ranges, threshold values, and/or measurements associated with a dataset. The dataset can comprise measurements associated with a population group (e.g., a population of similar age, gender, prior health conditions, environment, etc. to the subject) including alert/non-sedated individuals, sedated individuals, or a combination of the two. In other examples, the dataset can include historical data associated with the subject, such as measurements taken from the subject at a time when they were not intoxicated, drowsy, or otherwise sedated.

Regardless of the comparative ranges, threshold values, and/or datasets, divergence of the subject's motion measurements from data indicative of “normal” or non-sedated individuals can indicate sedative effects in the subject. Conversely, divergence of the subject's motion measurements from data indicative of sedation can indicate that the subject is not suffering from any sedative effects. In either case, convergences (as opposed to divergences) with the datasets could also be used to determine sedation in the subject.

Based on the foregoing comparisons and any detected divergences or convergences, in some examples, the PU can determine whether sedative effects are present in the user. In further examples, the PU can estimate a probability and severity of sedative effects on the user, optionally together with remediation options or instructions to halt current activity. The PU can then inform the user of the results of the assessment via the UI.

### Example 2

In another example, similar processes including some or all of the steps set forth in the previous example may be performed. Additionally or alternatively, the user can be asked or prompted to look at a light (e.g., light source 260 or an external, independent light source). In a further embodiment, the user can be asked or prompted to look at a blinking light. In either case, the PU can record the user's pupil and/or iris reaction to the light (whether blinking or steady) in the user's left and/or right eyes and these images can be used as set forth above to detect and assess intoxication, drowsiness, or other sedation.

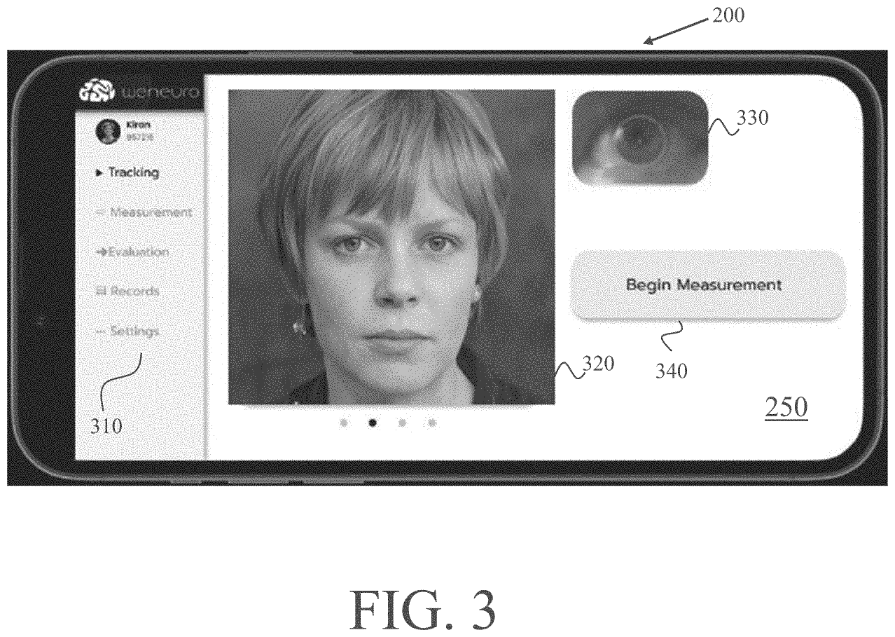

FIG. 3 depicts an illustrative example of UI/display 250 in use. In one aspect, the user is presented with progress bar 310. From progress bar 310, the user can track progress of a test as computing device 200 progresses from a “tracking” stage, to a “measurement” stage, to an “evaluation” stage. In one aspect, the tracking stage allows the user to confirm that computing device 200 has accurately located one or more areas of interest associated with the subject. For example, the UI in FIG. 3 depicts two images of the subject. A first image 320 depicts the user's entire face and a second image 330 depicts a zoomed in image of the subject's features relevant to testing. In the example shown, image 320 includes identifiers located on or around each of the subject's eyes. Likewise, image 330 depicts a zoomed-in image of one of the subject's eyes.

FIG. 3 further depicts an action button 340. If satisfied that computing device has accurately captured the relevant portions of the subject's face, the user can initiate the test by selecting action button 340.

FIG. 4 depicts another illustrative example of UI/display 250 in use. In one aspect, after the test is initiated, computing device 200 records and analyzes involuntary micromotions and/or fixational eye movements of the subject. In some examples, KLT feature tracking can be used to track image features (e.g., biomarkers) within a recorded video. For motion magnification, in some embodiments, a two-dimensional dual-tree complex wavelet transform can be used. The data collected with respect to the selected biomarkers over the duration of a test period (e.g., between 20 msec and 7000 msec) can then be analyzed.

In some examples, a live view of the video captured by the camera is displayed to the user at image 410. In further examples, within image 410, one or more biomarkers can be identified for tracking and analysis by, for example, KLT feature tracking. The UI can contain digital markings 420 superimposed on image 140 (e.g., depicted as small circles in image 140) to identify the selected biomarkers to the user.

In another aspect, the data acquired during the test (e.g., movement associated with one or more biomarkers) can be depicted and presented to the user in the form of graphs 430 and 440. Graphs 430 and 440 are only illustrative of examples of the type of measurements and visual representations that can be presented in the UI. For example, graph 430 can be a spectral power profile for one or both eyes in some embodiments. In another embodiment, graph 430 can depict pupil constriction, for instance, in degrees/time, XY-movement/time, and/or on a normalized scale of 0 to 1, for example.

In another example, graph 440 can depict movement of one or more biomarkers identified in a subject's eye(s) over time in some embodiments. In such embodiments, the x-axis can represent time and the y-axis can represent eye movement in degrees and/or XY-movement of the eye. In some embodiments, graph 400 can depict gaze direction, for example, in degrees/time and/or XY-movement (horizontal and vertical)/time.

In further examples, and similar to FIG. 3, the interface depicted in FIG. 4 can also include an action button 450. Upon completion of the testing cycle, the user can initiate the evaluation stage by selecting action button 450. In response to such a selection, in some embodiments, computing device will display to the user, via the UI, the results of the test.

Other embodiments of the systems and methods described here will be apparent to those skilled in the art from consideration of this specification and practice of the systems and methods disclosed herein. It is intended that the specification and examples be considered as illustrative only.

Though some of the described methods have been presented as a series of steps, it should be appreciated that one or more steps can occur simultaneously, in an overlapping fashion, or in a different order. The order of steps presented is only illustrative of the possibilities and those steps can be executed or performed in any suitable fashion. Moreover, the various features of the examples described here are not mutually exclusive. Rather any feature of any example described here can be incorporated into any other suitable example.

As used herein, the terms “sedation,” “sedative effects,” “intoxication,” “sleepiness,” and “drowsiness,” can be used interchangeably. Likewise, the terms “subject” and “affected individual” can be used interchangeably. The term “user” is intended to identify an individual operating computing device 200. The user may also be the subject or affected individual, or the user can use computing device 200 to diagnose and assess sedative effects in another individual who is the subject or affected individual.

Further, while computing device 200 and its components can be contained or integrated into in a single device, in other embodiments, computing device 200 can be comprised of multiple discrete devices, each containing one or more components of computing device 200 and each such discrete device being in communication with the other(s). For example, and without limiting other possibilities, one or more sensors 230 of computing device 200 (e.g., camera 232, accelerometer 234, and/or lidar 236) can be contained in a first discrete computing device and the remaining components (e.g., other sensors 230, processor 210, memory 220, display 250, light source 260, and/or database 270) can be contained in a second discrete computing device or distributed across multiple other discrete computing devices. Additionally, as discussed above, one or more components of computing device 200 (e.g., database 270) can be located in the cloud.

It is intended that the specification and examples be considered as exemplary only, with a true scope and spirit of the disclosure being indicated by the following claims.

What is claimed is:

1. A method for diagnosing and assessing sedative effects in a subject, comprising:
recording, by a camera, one or more videos of at least a portion of a subject's face;
identifying one or more biomarkers associated with the subject within each of the one or more videos;
measuring involuntary micromotions or fixational eye movements associated with each of the identified one or more biomarkers within each of the one or more videos;
comparing the measured involuntary micromotions or fixational eye movements associated with each of the identified one or more biomarkers to a predetermined value; and
based, at least in part, on the comparison, providing a notification indicating that sedation likely.

2. The method of claim 1, wherein the one or more videos of at least a portion of the subject's face comprises a first video including at least one of the subject's eyes.

3. The method of claim 1, wherein measuring the involuntary micromotions or fixational eye movements includes determining an amount of motion associated with the identified one or more biomarkers that is attributable to motion of the subject's head and taking the subject's head motion into account in determining the involuntary micromotions or fixational eye movements of the subject.

4. The method of claim 1, wherein the predetermined value is a value range indicative of values associated with a population comprising individuals not suffering from sedative effects.

5. The method of claim 4, wherein the population group comprises individuals of similar age, gender, prior health conditions, or environment as the subject.

6. The method of claim 1, wherein the dataset comprises historical data associated with the subject and collected at a time when the subject was not suffering from sedative effects.

7. The method of claim 1, wherein the comparison further includes determining a difference between the measured involuntary micromotions or fixational eye movements associated with each of the identified one or more biomarkers and the predetermined value, the difference exceeding a predetermined threshold.

8. A computing system for diagnosing and assessing brain injury in a subject, comprising:
a processor;
a memory; and
a camera,
wherein the processor performs stages including:
recording, by the camera, one or more videos of at least a portion of a subject's face;
identifying one or more biomarkers associated with the subject within each of the one or more videos;
measuring involuntary micromotions or fixational eye movements associated with each of the identified one or more biomarkers within each of the one or more videos;
comparing the measured involuntary micromotions or fixational eye movements associated with each of the identified one or more biomarkers to a predetermined value; and
based, at least in part, on the comparison, providing a notification indicating that sedation likely.

9. The computing device of claim 8, wherein the one or more videos of at least a portion of the subject's face comprises a first video including at least one of the subject's eyes.

10. The computing device of claim 8, wherein measuring the involuntary micromotions or fixational eye movements includes determining an amount of motion associated with the identified one or more biomarkers that is attributable to motion of the subject's head and taking the subject's head motion into account in determining the involuntary micromotions or fixational eye movements of the subject.

11. The computing device of claim 8, wherein the predetermined value is a value range indicative of values associated with a population comprising individuals not suffering from sedative effects.

12. The computing device of claim 11, wherein the population group comprises individuals of similar age, gender, prior health conditions, or environment as the subject.

13. The computing device of claim 8, wherein the dataset comprises historical data associated with the subject and collected at a time when the subject was not suffering from sedative effects.

14. The computing device of claim 8, wherein the comparison further includes determining a difference between the measured involuntary micromotions or fixational eye movements associated with each of the identified one or more biomarkers and the predetermined value, the difference exceeding a predetermined threshold.

15. A non-transitory, computer-readable medium comprising instructions that, when executed by a processor of a computing device, cause the processor to perform stages for diagnosing and assessing brain injury in a subject, the stages comprising:
recording, by a camera, one or more videos of at least a portion of a subject's face;
identifying one or more biomarkers associated with the subject within each of the one or more videos;
measuring involuntary micromotions or fixational eye movements associated with each of the identified one or more biomarkers within each of the one or more videos;
comparing the measured involuntary micromotions or fixational eye movements associated with each of the identified one or more biomarkers to a predetermined value; and
based, at least in part, on the comparison, providing a notification indicating that sedation likely.

16. The non-transitory, computer-readable medium of claim 15, wherein the one or more videos of at least a portion of the subject's face comprises a first video including at least one of the subject's eyes.

17. The non-transitory, computer-readable medium of claim 15, wherein measuring the involuntary micromotions or fixational eye movements includes determining an amount of motion associated with the identified one or more biomarkers that is attributable to motion of the subject's head and taking the subject's head motion into account in determining the involuntary micromotions or fixational eye movements of the subject.

18. The non-transitory, computer-readable medium of claim 15, wherein the predetermined value is a value range indicative of values associated with a population comprising individuals not suffering from sedative effects.

19. The non-transitory, computer-readable medium of claim 18, wherein the dataset comprises historical data associated with the subject and collected at a time when the subject was not suffering from sedative effects.

20. The non-transitory, computer-readable medium of claim 15, wherein the comparison further includes determining a difference between the measured involuntary micromotions or fixational eye movements associated with each of the identified one or more biomarkers and the predetermined value, the difference exceeding a predetermined threshold.
